(19) United States
(12) Patent Application Publication
(10) Pub. No.: US 2007/0189983 A1
(43) Pub. Date: Aug. 16, 2007

(54) Tooth whitening composition

(75) Inventors: Jennifer Jane Gordon, Surrey (GB); Jose Luis Velada, Surrey (GB)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(73) Assignee: Glaxo Group Limited

(21) Appl. No.: 10/586,214

(22) PCT Filed: Jan. 17, 2005
(86) PCT No.: PCT/GB05/00135

§ 371 Date: Jul. 17, 2006

(30) Foreign Application Priority Data
Jan. 19, 2004 (GB) 0401113.6

(51) Int. Cl. A61K 8/22

(52) U.S. Cl. 424/53

(57) ABSTRACT

The present invention provides an anhydrous liquid tooth whitening composition comprising a peroxide-containing compound and an orally acceptable carrier wherein the carrier includes a humectant, a bioadhesive agent, and a film-forming agent consisting essentially of a water-insoluble film-forming agent and a solvent for the film-forming agent.

The present invention relates to a tooth whitening composition for bleaching tooth enamel. Specifically the present invention relates to an anhydrous tooth whitening composition comprising a peroxide.

White teeth have long been considered cosmetically desirable. Unfortunately, teeth almost invariably become discoloured in the absence of intervention. The tooth structure which is generally responsible for presenting a stained appearance is the enamel layer. Several factors contribute to enamel discoloration, but the three main factors are believed to be: (i) formation of plaque and tartar matrices on the tooth surface which then entraps stains; (ii) ingestion of certain drugs during gestational tooth formation; and (iii) discoloration due to oral cavity traumatization following which blood break-down products seep into the mineralized area of the teeth during enamel formation. This invention is primarily concerned with the first factor of tooth discoloration, that is, the natural stain which accumulates on teeth.

Over-the-counter teeth whitening preparations have been developed to address the cosmetic preference of many to restore luster to tooth enamel discolored by surface entrapped materials. While all dentifrices and mouthwashes contain some cleaning and polishing agents, some enamel deposits become intractable to being fully removed by these agents under normal use conditions. For example, smokers often develop discolored enamel because the tars and particulate in exhaled cigarette smoke collect on the teeth. Further, a number of comestibles, such as tea, or some medicinal agents, can stain or discolor tooth enamel.

There are various approaches to enamel whitening currently in general use. One approach is a physical abrading of the stain to effect stain removal. Harsher abrasives, also known as polishing agents, than those used in typical non-whitening toothpaste preparations, are employed in this approach. Most, if not all of these preparations are toothpastes, gels or powder dentifrices. The mechanical process may be supplemented or even replaced by a chemical process which may involve an oxidative or enzymatic step to effect stain removal.

The chemical process generally utilizes a tooth whitening or bleaching formulation applied to a stained tooth surface for a specified period, after which the formulation is removed. Oxidizing agents represent one of the most widely distributed and utilized active agents in commercially available tooth whitening or bleaching products. Peroxide-containing agents such as carbamide peroxide, hydrogen peroxide and calcium peroxide are the most commonly used oxidizing agents, and are typically formulated into a liquid, solution, gel or paste. However it is known that products containing such agents may lose their whitening efficacy with time. In addition aqueous peroxide formulations may have only a brief period of efficacy when applied to teeth in the oral cavity because of rapid decomposition of peroxide on exposure to the enzyme, catalase, present in high concentrations in saliva. Moreover low viscosities of aqueous peroxide solutions do not allow the peroxide whitening agent to remain in contact with teeth for the necessary time period to effect substantive whitening because of constant flushing effects of salivary secretions. WO 03/099246 (Colgate-Palmolive) aims to address these problems with the provision of an aqueous tooth whitening liquid composition comprising an orally acceptable vehicle comprising water and monohydric alcohol having dispersed therein a film forming combination of a poly(ethylene oxide) and a carbomer. However there remains a need for alternatives that do not suffer drawbacks encountered with known formulations. Moreover it would be desirable to provide simple formulations that do not require extensive stabilization, that are easy to manufacture and are sufficiently substantive and robust to enable once-a-day application. In addition it would be highly desirable to provide a composition that is capable of producing an instant whitening effect on the teeth whilst the bleaching process is taking place.

It is an object of the present invention to provide a composition that meets these requirements. This object is met according to the invention by the provision of an anhydrous liquid tooth whitening composition comprising a peroxide-containing compound and an orally acceptable carrier wherein the carrier includes a humectant, a bioadhesive agent, and a film-forming agent consisting essentially of a water-insoluble film-forming agent and a solvent for the film-forming agent.

The composition of the invention is essentially anhydrous. Water or an aqueous medium is not used as a carrier or vehicle in the composition. Whilst free water is not added to the composition, it will be understood that small amounts of water, i.e. less than 5% w/w, preferably less than 3% w/w, may be present as a result of being introduced with other materials e.g. by using a “stock” hydrogen peroxide aqueous solution such as a 30% w/w hydrogen peroxide solution.

The composition of the invention is suitably in the form of a varnish that may conveniently be painted onto the teeth. Following its application, the composition dries in situ to form an adherent film which sticks firmly to the teeth. The film is sufficiently strong and adherent to remain on the teeth for a period of time e.g. up to a few hours or even longer e.g. up to twelve hours or more. Suitably the composition is applied at night time, allowing peroxide activity to take place during sleep periods. During use, the peroxide is released slowly from the film, in an amount and at such a rate as required to effect stain removal. The film may be removed from the user's teeth by any convenient means e.g. brushing and/or by rinsing with a mouthwash.

The term “painted onto the teeth” above is intended to encompass all manner of applying the whitening composition to teeth and includes sponging, coating, daubing, spraying, wiping and rubbing. Preferably the composition is applied to the teeth with a soft applicator brush. Suitably the composition is housed in a container such as a bottle, and the composition is applied with a soft applicator brush. The bottle and brush may be provided in kit form e.g. as may be used with a conventional nail varnish kit. Alternatively the composition may be housed in a dispensing device such as a pen with an applicator brush attached thereto e.g. generally of the type used with the Brite Smile To Go™ Whitening pen.

Peroxide-containing compounds used as whitening agents in the present invention include the following compounds and mixtures thereof: hydrogen peroxide e.g. as 30% w/w aqueous solution, carbamide peroxide, calcium peroxide, percarbonates and hydrogen peroxide polymer complexes for example hydrogen peroxide complexes with solid linear or crosslinked poly(-vinyl-pyrrolidone) (PVP) homopolymers and its copolymers, such as Peroxydone™ K30, Peroxydone™ K90, Peroxydone™ XL10; and hydrogen peroxide complexes with copolymers of vinyl-pyrrolidone and vinyl acetate, such as Plasdone® S-630. These various Peroxydone™0 and Plasdone® polymers are available from International Specialty Products (ISP), 1361 Alps Road, Wayne, N.J. 07470, US. Insoluble crosslinked polymeric matrices that retain hydrogen peroxide for a certain period of time may also be used. An example of this is Poly-Pore® 337HP which is an allyl methacrylate crosspolymer and is available from AMCOL Health & Beauty Solutions, Inc., 301 Laser Lane, Lafayette, La. 70507, US.

Mixtures of different peroxide sources, as hereinabove described, may be used to provide variable peroxide release. For example both immediate and slow release peroxide may be achieved by using a combination of aqueous hydrogen peroxide and carbamide peroxide and/or hydrogen peroxide complexes with solid vinyl-pyrrolidone (VP) polymers. Carbamide peroxide, and mixtures of carbamide peroxide with hydrogen peroxide (30% aqueous solution) and optionally with a hydrogen peroxide complex with vinyl-pyrrolidone e.g. Peroxydone™ hydrogen peroxide polymer complexes e.g. Peroxydone™ K90, are preferred.

Suitably the total amount of peroxide present in the liquid whitening composition of the invention is in the range of 1 to 30% w/w, preferably in the range 2 to 20% w/w, even more preferably in the range 3 to 10% w/w.

A humectant is a key component of a composition of the invention. It has been found that the absence of a humectant results in the formation of brittle and fragile films that cannot be adequately spread onto teeth. Suitable humectants include the following and mixtures thereof: glycerin, sorbitol, propylene glycol, sugars such as glucose or sucrose, and low molecular weight polyethylene glycols (PEGs) i.e. in the range 200-600 e.g. PEG 200, 300, 400, and 600, available from Dow Chemicals USA, PO Box 1206, Midland Mich. 48642. Preferred humectants include glycerin and PEGs.

Suitably the humectant is present in an amount ranging from 0.5 to 30% w/w, preferably in the range 1 to 15% w/w.

A bioadhesive agent is included in a composition of the invention. The bioadhesive agent enhances substantivity of the composition to teeth. Suitably the bioadhesive agent of the invention exhibits mucoadhesive behaviour i.e. it has an affinity for biological surfaces such as teeth. Examples of suitable bioadhesive agents include carbomers, copolymers of methyl vinyl ether and maleic anhydride, natural gums, vinyl-pyrrolidone polymers and copolymers and mixtures thereof.

Carbomers are synthetic high molecular weight polymers of acrylic acid that are crosslinked with either allylsucrose or allyletheres of pentaerythritol. Carbomers sold under the trade name “Carbopol®”, available from Noveon Inc, 9911 Brecksville Road, Cleveland, Ohio 44141-3247, are preferred and include Carbopol® 934 or 974, 940, 941, 980 and Ultrez 10™. Other suitable carbomers include partially neutralized carbomers e.g. PNC400, available from 3V Sigma, PO Box 219, Via Torquato Tasso, 58,24100, Bergamo, Italy and carbomer copolymers such as crosslinked copolymers of acrylic acid with alkylacrylate where the alkyl chain is C10-30 e.g. Pemulen TR1 and Pemulen TR2, available from Noveon Inc. as above.

Copolymers of methyl vinyl ether and maleic anhydride are available commercially in a range of molecular weights under the trade name “Gantrez®” (ISP), specifically Gantrez® AN. Other Gantrez® copolymers that may be used include the free acid form of the Gantrez® AN available as Gantrez® S, a mixed sodium and calcium salt of Gantrez® S available as Gantrez® MS, and half ester derivatives of Gantrez® S available as Gantrez® ES.

Natural gums such as gum karaya, xanthan gum, guar gum, arabic gum tragacanth are also suitable bioadhesive agents. Xanthan gum is especially preferred and has been found to impart surprisingly good substantivity properties to compositions of the invention.

Suitable vinyl-pyrrolidone polymers include poly(-vinyl-pyrrolidone) (PVP) and cross-linked PVP. A suitable copolymer as hereinbefore described includes Plasdone® S-630. PVP is a preferred polymer, in particular a high molecular weight PVP e.g. in the range 1,300,000 e.g. Plasdone® K-90.

Preferably the bioadhesive agent is selected from xanthan gum, a carbomer and PVP and mixtures thereof. Even more preferably the bioadhesive agent is a mixture of a carbomer such as a Carbopol and a high molecular weight PVP e.g. Plasdone® K-90.

Suitably the bioadhesive agent is present in an amount ranging from 0.5 to 30% w/w, preferably from 1 to 20% w/w.

On application to teeth, the film-forming component of the composition forms a protective film or barrier which retains the other components, i.e. excipients and the peroxide-containing compound, of the composition in a matrix-type environment. The only component which is not retained is the solvent for the film-forming agent which evaporates from the site of application. Suitably the film-forming component of the composition consists essentially of a water-insoluble film-forming agent such as a water-insoluble microcrystalline cellulose, e.g. ethyl cellulose, a poly(urethane) or a poly(acrylamide). Ethyl cellulose is a preferred film-forming agent. Suitably the film-forming component is present in an amount ranging from 5 to 30% preferably 10 to 25% w/w.

A solvent is required for the film-forming agent. The solvent is a carrier for the film-forming agent. During use the solvent rapidly evaporates to leave a highly substantive film on the teeth. The deposited film is comprised of a peroxide, a humectant, a bioadhesive agent and a water-insoluble film-forming agent. Examples of suitable solvents include monohydric alcohols such as ethanol or isopropyl alcohol. Suitably the solvent is present in an amount ranging from 30 to 80%, preferably 40 to 70% w/w.

Additionally, compositions of the present invention will suitably contain acceptable additives or excipients conventional in the field of oral care products including for example chelating agents such as ethylenediaminetetraacetic acid and/or citric acid, colouring agents, flavouring agents, a fluoride source such as sodium fluoride or sodium monofluorophosphate, an antisensitivity agent such as strontium or potassium salts e.g. strontium chloride, and sweetening agents. The additives or excipients used in any given composition will be compatible both with each other and with the essential ingredients of the composition such that there is no interaction which would impair the performance of the active ingredients. All additives or excipients must of course be non-toxic and of sufficient purity to render them suitable for human use.

The liquid whitening compositions of the present invention are prepared by adding and mixing the ingredients of the composition in a suitable vessel such as a stainless steel tank provided with a mixer. In the preparation of the composition the ingredients are suitably added to the mixer in the following order: peroxide, chelating agents (if used), humectant, solvent for the film-forming agent to form a solution. The film-forming agent is then added, followed by addition of the bioadhesive agent. The whitening composition prepared is then suitably packaged and stored as required.

Advantageously the composition of the invention is suitably prepared in the form of a “single component” system i.e. all components of the whitening composition, i.e. excipients and the peroxide-containing compound, are self-contained in a desired pre-mixed proportion. Accordingly there is no requirement for the components of the composition to be physically separated from each other prior to use in order to avoid any undesirable interactions, as may occur with some peroxide-containing formulations. In a second aspect of the invention there is provided a kit of parts comprising an anhydrous liquid tooth whitening composition as hereinbefore described, a container for housing the composition and an applicator for applying the composition to teeth to be whitened.

Compositions according to the present invention may be applied topically to the teeth as appropriate in the form of lotions, gels, mousses, sprays or aerosols.

In an alternative aspect there is provided a method of whitening teeth comprising:
a. preparing a tooth whitening composition as hereinbefore described;
b. painting the composition onto teeth to be whitened, suitably at night-time
c. maintaining the composition in contact with the teeth for a plurality of hours per day e.g. up to twelve hours per day,
d. repeating steps b and c for multiple days e.g. up to fourteen days, to thereby whiten the teeth.

The present invention is illustrated by the following examples but is not limited thereby.

### Example 1

A whitening composition was prepared having the following ingredients:

| Ingredient | % w/w |
|---|---|
| Glycerin | 3.00 |
| Ethanol | 67.00 |
| Carbamide peroxide | 3.00 |
| Hydrogen peroxide (30%) | 3.00 |
| Peroxydone K-90 | 10.00 |
| Ethyl cellulose | 14.00 |
| Total | 100.00 |

### Example 2

A whitening composition was prepared having the following ingredients:

| Ingredient | % w/w |
|---|---|
| Glycerin | 4.00 |
| Ethanol | 66.00 |
| Carbamide peroxide | 3.00 |
| Hydrogen peroxide | 3.00 |
| Peroxydone K-90 | 11.00 |
| Xanthan gum | 2.00 |
| Ethyl cellulose | 11.00 |
| Total | 100.00 |

### Example 3

A whitening composition was prepared having the following ingredients:

| Ingredient | % w/w |
|---|---|
| Carbamide peroxide | 17.00 |
| Ethanol | 45.00 |
| Ethyl cellulose | 15.00 |
| Carbopol 974P NF | 1.00 |
| Glycerin | 22.00 |
| Total | 100.00 |

### Example 4

A whitening composition was prepared having the following ingredients:

| Ingredient | % w/w |
|---|---|
| Carbamide peroxide | 5.60 |
| Hydrogen peroxide (30%) | 6.90 |
| Ethanol | 62.10 |
| Ethyl cellulose | 11.30 |
| Plasdone K-90 | 9.00 |
| Xanthan gum | 2.20 |
| Glycerin | 2.80 |
| Citric Acid | 0.05 |
| EDTA | 0.05 |
| Total | 100.00 |

### Example 5

An instant whitening composition was prepared having the following ingredients:

| Ingredient | % w/w |
|---|---|
| Ethanol | 65.00 |
| Ethyl cellulose | 5.00 |
| Peroxydone K-90 | 22.90 |
| Mica | 2.00 |
| Glycerin | 4.00 |
| Titanium dioxide | 1.00 |
| Citric Acid | 0.05 |
| EDTA | 0.05 |
| Total | 100.00 |

### Example 6

A whitening aerosol spray composition was prepared having the following ingredients:

| Ingredient | % w/w |
|---|---|
| Ethanol | 45.00 |
| Hydrogen peroxide (30%) | 10.00 |
| Ethyl cellulose | 5.00 |
| Peroxydone XL10 | 10.00 |
| Glycerin | 5.00 |
| DME | 25.00 |
| Total | 100.00 |

### Example 7

A whitening aerosol spray composition was prepared having the following ingredients:

| Ingredient | % w/w |
|---|---|
| Ethanol | 45.00 |
| Hydrogen peroxide (30%) | 15.00 |
| Ethyl cellulose | 8.00 |
| Peroxydone XL10 | 2.00 |
| Glycerin | 5.00 |
| DME | 25.00 |
| Total | 100.00 |

Determination of Bleaching Effects using Bovine Enamel Night Time Whitening Model
Introduction

The bleaching effects of compositions disclosed in Examples 1-3 above were determined and compared to the bleaching effects observed with a commercially available preparation (a whitening product available as “Crest Night Effects”).

Method

Bovine teeth were used and L* (from the CIE 1976 L*a*b* colour space scale) was measured at the start of the experiment using a spectrocolorimeter. Formulations were applied to the teeth and these were placed into a container such that a liquid substantivity challenge was applied to the treated teeth. At the end of the treatment time the teeth were rinsed and dried and L* was remeasured using a spectrocolorimeter. This method was repeated for a number of days to mimic in-vivo nighttime use of the product. At the end of the experiment the overall change in L* i.e. ΔL was calculated.

Results

| Sample | ΔL after 12 hrs treatment |
|---|---|
| Example 1 | 4.49 |
| Example 2 | 5.55 |
| Example 3 | 3.31 |
| Crest Night Effects | 4.02 |
| Deionised water | −1.83 |

Conclusions

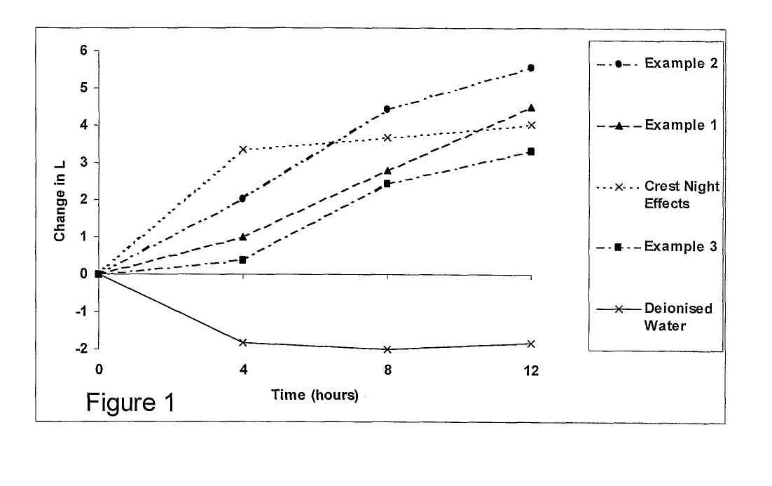

The results indicate that change in ‘L’ after 12 hours treatment was comparable to that observed with a commercially available nighttime product. The results are illustrated graphically in FIG. 1 on page 1/1.

A further randomized, single centre, parallel and examiner-blind study was undertaken.

Introduction

Healthy subjects with a Vita Shade Guide value of A2 or grater on two of the 4 maxillary incisors were enrolled. The maxilliary anterior teeth were treated for 14 days at home by applying the treatment for 30 seconds once daily after brushing at night. The treatment groups were:
(1) Example 4 Formulation
(2) Commercial paint-on product containing 10% hydrogen peroxide
(3) Brushing twice daily alone
Method
All subjects were requested to refrain from eating, drinking and smoking for 30 minutes after the gels were applied. All subjects brushed with the same dentifrice twice daily using the same make of toothbrush for one minute using a timer. To follow the change in intrinsic tooth colour, shade assessments were performed at baseline and after 7 and 14 days of treatment. An analysis of covariance (ANCOVA) model using the baseline mean subject-wise Vita Shade score as covariate including treatment group, age group and smoking status was used.

Results

| | | Commercial | Brushing twice |
|---|---|---|---|
| | Example 4 | Product | daily alone |
| No. subjects | 32 | 32 | 32 |
| Baseline Mean | 7.98 | 8.12 | 8.81 |
| 7 days | 1.09 | 1.33 | −0.05 |
| 14 days | 2.21 | 2.14 | 0.00 |

Conclusion

The results show that a significant change in shade was observed after 14 days treatment with the Example 4 formulation.

1. An anhydrous liquid tooth whitening composition comprising a peroxide-containing compound and an orally acceptable carrier wherein the carrier includes a humectant, a bioadhesive agent, and a film-forming component consisting essentially of a water-insoluble film-forming agent and a solvent for the film-forming agent.

2. An anhydrous liquid tooth whitening composition according to claim 1 wherein the peroxide is selected from carbamide peroxide, and mixtures of carbamide peroxide with hydrogen peroxide and/or a hydrogen peroxide complex with vinyl pyrrolidone.

3. An anhydrous liquid tooth whitening composition according to claim 1 wherein the humectant is glycerin.

4. An anhydrous liquid tooth whitening composition according to claim 1 wherein the bioadhesive agent is selected from xanthan gum, a carbomer and PVP and its copolymers and mixtures thereof.

5. An anhydrous liquid tooth whitening composition according to claim 1 wherein the film forming agent is ethyl cellulose.

6. An anhydrous liquid tooth whitening composition according to claim 1 wherein the solvent is ethanol.

7. A kit of parts comprising an anhydrous liquid tooth whitening composition according to any claim 1, a container for housing the composition and an applicator for applying the composition to teeth to be whitened.

8. A method of whitening teeth comprising preparing a tooth whitening composition according to claim 1;
(a) painting the composition onto teeth to be whitened;
(b) maintaining the composition in contact with the teeth for a plurality of hours per day; and then
(c) repeating steps b and c for multiple days to thereby whiten the teeth.
